(19) United States
(12) Patent Application Publication
(10) Pub. No.: US 2009/0286309 A1
(43) Pub. Date: Nov. 19, 2009

(54) Device for producing a biologically active substance

(75) Inventor: Enno Roderfeld, Pfullendorf (DE)

Correspondence Address: POSZ LAW GROUP, PLC, 12040 SOUTH LAKES DRIVE, SUITE 101, RESTON, VA, 20191, US

(73) Assignee: Roderfeld und Bora GmbH & Co. KG, Uberlingen (DE)

(21) Appl. No.: 12/314,864

(22) Filed: Dec. 18, 2008

(30) Foreign Application Priority Data
May 14, 2008 (DE) 10 2008 023 545.8

(51) Int. Cl. C12M 1/04

(52) U.S. Cl. 435/303.1

(57) ABSTRACT

A device, a method and a system that are used for producing at least one biologically active substance. The device has at least one unit (12) with a main body (14).

### CROSS REFERENCE TO RELATED APPLICATION

This application is based on and incorporates by reference German Patent Application No. DE 10 2008 023 545.8, which was filed on 14 May 2008.

### BACKGROUND OF THE INVENTION

The invention relates to a device which is used for producing at least one biologically active substance and which has at least one unit with a main body.

### SUMMARY OF THE INVENTION

It is proposed that the unit is a container. Here, a process of “production” refers in particular to production, recovery, enrichment and/or multiplication of a substance, such as a biologically active substance. A “biologically active substance” is to be understood herein as a substance and/or a product that has an influence, for example a stimulation and/or an inhibition of a production and/or of a process, and/or another influence, appearing useful to a person skilled in the art, on an element and/or a system, in particular on a biological system. Here, a biological system is in particular an organism and/or a living being, such as a microorganism (bacteria, fungi, algae, protozoa, etc.), a plant, or an animal, but it is also to be understood as other elements appearing useful to a person skilled in the art, for example a virus, and/or systems. An influence or a mechanism of action of the substance is here preferably based on an interaction of the substance with at least one component of the organism, e.g. ions, trace elements, nucleic acids, amino acids, peptides, proteins (hormones, enzymes, products of metabolism, products of degradation, etc.) and/or at least one exogenous substance present in the organism, e.g. xenobiotics (medicaments, drugs, toxins, environmental poisons and/or food additives, etc.). In this connection, a “unit” is to be understood in particular as a structure made up of several components, for example a main body, which form a functional unit and/or which can be separated from one another only with the function of the unit being lost. A “main body” preferably forms a principal component of the unit and thus defines in particular a form and/or shape of the unit. This form is preferably tubular, in the form of a cylinder jacket, with an end face at one end at least of an axial main extent of the cylinder jacket, for example a conically extending bottom part. However, other forms appearing expedient to a person skilled in the art would also be possible. The main body is preferably made of metal, glass and/or particularly advantageously of a plastic, for example polystyrene, polypropylene, polycarbonate, polyethylene, polyvinyl chloride, polyethylene terephthalate and/or another material appearing expedient to a person skilled in the art, in particular a material that does not impair the production of the biologically active substance. A “container” is to be understood herein as a receptacle that is designed differently than a syringe and whose main body forms in particular, at least for the most part, compared in particular to a bag, rigid receptacle walls. Moreover, in at least one operating state in particular, the container should be tight or impermeable with respect to its environment and should protect its content from the effects of the environment and/or its surroundings. By virtue of the design of the unit as container, the device can be made particularly stable, secure and operator-friendly. Good handling, with minimizing of errors, is ensured in particular by the possibility of uncomplicated and safe transport.

It is also proposed that the container has a wing-free design. In this connection, “wing-free” is to be understood as a configuration of the container, and in particular, a configuration of a surface of the cylinder jacket, without an integrally formed or attached wing normally extending in a direction perpendicular to the main direction of orientation of the cylinder jacket by at least one finger's width or by at least 5 mm, for fixing a position of a receptacle. In this way, a compact structure can be achieved which is advantageously suitable for compact storage and for good transport, for example, of several containers together.

It is also advantageous if the container has a pistonless design. Here, “pistonless” is to be understood as a configuration of the container, and in particular, a configuration of a cavity enclosed by the cylinder wall, without a piston or plunger normally arranged movably in the main direction of orientation of the cylinder jacket and in the cavity enclosed by the cylinder jacket for generating a pressure for drawing in and/or discharging gases and/or liquids into and/or out of a cavity of a receptacle. By virtue of this special configuration, a particularly stable device can be made available which, in addition, is inexpensive and easy to produce.

In another embodiment of the invention, it is proposed that at least one closure device be provided which is connected irreversibly to the main body. Here, a “closure device” is to be understood in particular as a device, such as a cover in the form of a lid or cap, that closes and/or seals off the main body in at least one operating state, for example, in a transportation situation, against the entry and/or discharge of a gas, for example air, a liquid and/or a solid.

Moreover, “irreversibly” is to be understood as meaning that the closure device can be separated from the main body only with loss of function of the device itself. It is conceivable to use all types of form-fit, force-fit and/or cohesive connections that appear useful to a person skilled in the art, for example locking, gluing, welding, sintering and/or soldering, for connecting the closure device to the main body. It would also be possible, however, for the closure device to be designed in one piece with the main body. The closure device is preferably made of glass, rubber, a plastic, for example polystyrene, polypropylene, polycarbonate, polyethylene, polyvinyl chloride, polyethylene terephthalate, and/or particularly advantageously of metal and/or another material appearing expedient to a person skilled in the art, in particular a material that does not impair the production of the biologically active substance. The closure device is preferably arranged, along the main direction of orientation of the cylinder jacket, at an end remote from the end face of the main body. By means of the closure device, the device itself can be made particularly safe to use and operator-friendly and, furthermore, a condition that prevails in a cavity enclosed by the cylinder jacket surface, for example an underpressure or a sterility, can be particularly easily maintained.

It is further proposed that the device has at least one pierceable membrane. In this connection, a “pierceable membrane” is to be understood in particular as a part of the unit, of the main body and/or of the closure device that is made of a material which, in terms of strength, elasticity, permeability and/or another property appearing useful to a person skilled in the art, is of a different nature than that of another part of the unit, of the main body and/or of the closure device, for example a cover area of the closure device. This material is preferably a material that is at least substantially impermeable to gases, solids and/or liquids, for example rubber, plastic and/or silicone, and that can be easily pierced by a sharp object, such as a cannula, which can, for example, have an external diameter of between 1.5 and 0.5 mm (17 to 25 gauge) and a length of between 20 and 100 mm. However, it would also be conceivable to use another material appearing expedient to a person skilled in the art, for example cellulose or a derivative thereof. The pierceable membrane is particularly preferably arranged in the closure device and, in particular, in a recess of the closure device. The pierceable membrane is a structurally simple and user-friendly way of allowing material, such as liquid and/or gas, to be removed from a cavity of the container, in particular in a sterile manner. Moreover, a sterility, at least of the pierceable membrane arranged in the closure device, can be advantageously ensured by simple structural means if the closure device has a cover unit, for example a pull-off film, preferably made of plastic.

It can also be advantageous if the device has at least one gas outlet valve and/or one gas inlet valve. In this connection, a “gas inlet valve” or “gas outlet valve” is to be understood in particular as a technical component that serves to control an outflow and/or inflow of gases, in particular air, and/or to control and/or regulate a direction of flow. The gas outlet valve can be opened in order to avoid an overpressure, such that when a cavity of the container is filled with a liquid, the gases present in the cavity are allowed to escape. The gas inlet valve, by contrast, serves for better removal of the liquid from the container, in order to prevent an underpressure in the cavity during the removal of liquid. The gas outlet valve and/or gas inlet valve are preferably arranged in the closure device, although an arrangement in the main body would in principle be possible. The gas outlet valve and/or gas inlet valve can be formed in particular by a straight-way valve, a shutoff valve, a nonreturn valve, a roller diaphragm valve and/or another valve appearing useful to a person skilled in the art, and it can be actuated manually, mechanically, electromagnetically, electrically, hydraulically, pneumatically and/or by means of a medium such as gas, liquid and/or another medium, and/or in some other way. The gas outlet valve and/or gas inlet valve is advantageously designed as a nonreturn valve and in particular has a diaphragm. However, it would also be possible for the valve to be designed with openings that can be freed by manual rotation of a locking ring. An efficient utilization of the space in the closure device can be achieved if the pierceable membrane is arranged between the gas outlet valve and the gas inlet valve. Here, the expression “between the gas outlet valve and the gas inlet valve” is to be understood in particular as a spatial arrangement with respect to the gas outlet valve and the gas inlet valve. The configuration of the gas outlet valve and/or gas inlet valve is a simple way, in structural terms, of providing a pressure equalization for the cavity of the container.

If the container has a cavity in which, in at least one operating state, there is a pressure deviating from an atmospheric pressure, a good delivery of blood or of blood constituents into the cavity can easily be achieved by means of a blood sampling device which is attached via a connection unit. Here, the operating state is preferably an operating state in which the cavity is completely empty of liquid. In the case of the device, this is the operating state before the liquid blood constituents flow into the cavity. An atmospheric pressure here defines an average air pressure of the atmosphere, which, for example, is 101325 Pa at sea level. A pressure deviating from this represents an underpressure and/or a vacuum. In this embodiment, the number of parts can be reduced by dispensing with a gas outlet valve.

At least one connection unit is advantageously arranged on the main body. Here, a “connection unit” is to be understood in particular as a unit which connects the main body of the container to another functional unit, such as a blood-sampling device, for example in the form of a syringe, a blood bag and/or an infusion tube. The connection unit can be a connection tube, but it is preferably formed by what is called a Luer cone, and particularly preferably by what is called a Luer lock. The connection unit is preferably arranged on the end face of the main body. However, an arrangement at another location would also be conceivable. The connection unit is preferably designed in one piece with the main body, the expression “in one piece” meaning in particular that the closure device can be separated from the main body only with loss of function. In principle, however, a sleeve nut of the Luer lock could also be designed as a separate component, which can then be mounted with a form fit onto the Luer cone formed integrally on the main body. The arrangement of the connection unit is a structurally simple way of allowing substances to be delivered to and removed from the cavity of the container. Moreover, the connection unit can have a closure cap, which is provided for the purpose of closing the connection unit, as a result of which a container can be made available which is operator-friendly and can be used in a versatile way in various process steps.

It is also proposed that the device has at least one cavity provided for the sterile storage of blood constituents. Here, a “cavity” is to be understood in particular as a space which is provided for storage of a substance, such as a solid, a gas and in particular a liquid, and particularly advantageously for the storage of blood and/or of blood constituents, and/or which serves in particular to isolate its contents from its environment. Here, the blood constituents originate from blood, in particular from whole blood. Here, the blood constituents define, on the one hand, a coagulum, accordingly cellular constituents of the blood (red and white blood cells), blood platelets and clotting factors and, on the other hand, a blood serum, in particular a blood sample without coagulum. The blood serum contains ca. 91% water and 7% proteins (albumins and globulins), the remaining 2% being made up of electrolytes, nutrients and hormones. In this connection, “sterile storage” is to be understood in particular as meaning that substances are introduced and/or stored under conditions in which the used materials, such as the container, the tubes, the cannulas or other articles considered necessary by a person skilled in the art, are free of nucleic acids and/or fragments thereof, proteins, viruses, living organisms, such as microorganisms, and/or their spores, and/or other contaminating substances. The sterilization is preferably done here by gamma radiation. The cavity and the sterile storage mean that a substance delivered to the device can be stored by structurally simple means, and a risk of contamination of the substance can advantageously be avoided.

A simple function can advantageously be achieved if the device has at least one shearing body. In this connection, a “shearing body” is to be understood in particular as a body and/or a structure which is provided for application of a shearing action or shearing forces to a sample, for example blood cells. Here, shearing defines in particular a deformation of the sample under the effect of a force that acts tangentially to a side surface of the sample. The deformation results in a stress situation for the sample or blood cells, whereupon the blood cells produce and/or release a biologically active substance. Thus, the arrangement and use of shearing bodies can permit a simple, effective and inexpensive stimulation of a production of the biologically active substance, for example products of metabolism. These products of metabolism and the container with the shearing bodies are intended preferably for use in the area of the treatment of inflammations. For this purpose, a collected blood serum, enriched with the product of metabolism, is intended to be introduced or injected in particular into an inflamed area, such as all the joints of the body, areas near the spinal column, perineural areas, muscles, tendons, ligaments, fascias and/or other areas that appear expedient to a person skilled in the art. It is not suitable for application directly into the blood stream, that is to say into veins and arteries and into a lymph system.

The products of metabolism that are produced are preferably involved in inflammatory reactions. The container can thus be used to produce and/or recover anti-inflammatory active substances. A preferred anti-inflammatory active substance is an interleukin-1 receptor antagonist (IL-1Ra). The interleukin-1 receptor antagonist is a receptor antagonist that specifically neutralizes an action of a protein, interleukin-1 (IL1). Interleukin-1, as a pro-inflammatory key cytokine, is one of the most important pro-inflammatory signal substances of the immune system. It is produced especially by monocytes and macrophages following an inflammation stimulus. Inflammation stimuli can be bacteria, fungi, such as yeasts, autoantigens, and any other kind of foreign particles. The application of the shearing forces to the blood cells by means of the shearing bodies also stimulates an inflammatory reaction. The pro-inflammatory effects of interleukin-1 are mediated by binding to an interleukin-1 receptor specific to interleukin-1. The interleukin-1 receptor antagonist is also released by immune cells and occurs in the body as a natural inhibitor of interleukin-1. It plays an important role in regulating an inflammatory process caused by interleukin-1. An effect of the interleukin-1 receptor antagonist is based on its slowing down or bringing to an end an action of the interleukin-1, by virtue of the fact that instead of interleukin-1 it itself docks onto the interleukin-1 receptor expressed by a target cell and thus prevents attachment of the interleukin-1, as a result of which the interleukin-1 can no longer deploy its pro-inflammatory effects. The interleukin-1 receptor antagonist is therefore the anti-inflammatory opponent of interleukin-1.

In a preferred development, the at least one shearing body is arranged in the at least one cavity, as a result of which a simple function of the device or of the container can be provided, which ensures that the sample or blood cells can be delivered, by simple structural means, to the shearing body generating the shearing forces.

It is also proposed that the at least one shearing body is formed by a bead. Here, a “bead” is to be understood preferably as a round, spherical body. By means of a bead shape, it is possible to provide a particularly efficient and also gently acting shearing body. In principle, the shearing body could also be formed by another structure that appears useful to a person skilled in the art. Such a structure could, for example, involve a profiling, a roughening and/or another modification of a surface of and/or in the main body. An increase in the size of this surface of the main body could also be made possible by means of a labyrinth system which is introduced into the cavity of the main body and through which the blood and/or blood constituents pass. However, the shearing and/or stressing of the blood cells could also be brought about by another process, for example by agitation of the container and, therefore, of the blood cells.

The device particularly advantageously has 240 shearing bodies in the form of beads. The beads can also be present in another number that appears useful to a person skilled in the art. The shearing body here has a diameter of between 2 and 5 mm, preferably of 4 mm, although here too it would be possible to have another diameter that appears expedient to a person skilled in the art. The configuration of the shearing body as a bead permits a particularly space-saving arrangement, in particular of a plurality of beads, in the cavity.

The shearing body or bead can be made of a metal, a plastic and particularly advantageously of glass, resulting in a shearing body that is easy to produce, light and inexpensive, which directly permits the provision of an inexpensive and operator-friendly device or container.

It is also advantageous if the shearing body has a smooth surface. A “smooth surface” is to be understood in particular as a surface which has elevations and/or depressions with an orientation deviating from the main orientation of the surface, the elevations and depressions being less than 0.1 mm. This configuration permits gentle and efficient treatment of the blood or the blood constituents, such as blood cells, by which the cells can be exposed to a moderate stress, but one that does not lead to destruction of the cellular structures, for example the cell membrane.

It is further proposed that the at least one cavity has a capacity of greater than 50 ml, advantageously of between 50 and 100 ml, and particularly preferably of between 50 and 65 ml. In general, however, another capacity of between 10 and 100 ml would also be conceivable. Here, a “capacity” is to be understood in particular as the maximum volume that can be held by a body such as a cavity. The material filling it can be solid, liquid and/or gaseous. If a volume of blood of 50 ml is withdrawn from a patient, a treatment of the blood with the shearing bodies yields an interleukin-1 receptor antagonist concentration up to 140 times greater than the concentration in an untreated sample. In this process, an efficient enrichment of an active substance can easily be achieved in a useful volume. In addition, a sufficient volume is present to permit portioning into smaller sample volumes. These portioned samples can be stored and can be used at a later time. Moreover, blood only has to be taken once from the patient, and this, particularly when it is an animal that is the patient, protects and makes matters easier for an operator who is taking the sample of blood. Moreover, using the patient's own blood permits a quick method of obtaining the active substance.

In another embodiment, a system for producing at least one biologically active substance with at least one device is proposed, in which case at least one further means for blood sampling and/or blood preparation is provided, in addition to a container, as a component of the system. Here, a “means for blood sampling and/or blood preparation” is to be understood in particular as an article, for example a syringe, a cannula, a butterfly, a closure cap, a connection tube, a connection device, a sterilizing item, a centrifugation support, operating instructions and/or another article which appears useful to a person skilled in the art and which can be used in a process of blood sampling and/or blood preparation. Here, “blood sampling” defines in particular the collection of a blood sample, preferably from a blood vessel, for example an artery, a vein and/or a jugular vein, of a mammal. It is collected preferably by means of a cannula, the term “cannula” also being understood here as a butterfly cannula, an indwelling cannula and/or another blood-sampling means that appears expedient to a person skilled in the art. A suitable mammal for this purpose is in particular a human, a monkey, a dog, a cat, a perissodactyl, such as a horse, an artiodactyl, such as a camel, a cow and/or a pig, or a proboscoid, such as an elephant. However, it would also be conceivable for blood to be taken from another animal organism. Here, “blood preparation” is to be understood in particular as a process in which collected blood and/or blood constituents are subjected to a treatment process. This treatment process preferably includes delivery of the blood to the container or into the cavity of the container, centrifugation of the container, if appropriate with a centrifugation support specially adapted to the container, and/or removal and/or storage of blood constituents from the container. However, other process steps that appear expedient to a person skilled in the art could also be foreseen. By provision of a system including a further means, a complete package can easily be made available which is particularly operator-friendly and which means that a user, for example a physician, does not have to assemble the necessary components for carrying out the process. It can also be ensured that all the necessary components are immediately to hand during the blood sampling and/or blood preparation.

A centrifugation support for a container is also provided whose shape is adapted at least to an outer shape of the container. In this connection, a “centrifugation support” is to be understood in particular as a means, such as a centrifugation plate or in particular a centrifugation tube and/or another means which appears expedient to a person skilled in the art, and which can be used for a centrifugation procedure and, in particular, bears and/or supports an article, for example the container, in a centrifuge during the centrifugation procedure. Here, the “outer shape” of the container defines in particular an outer contour of the container which is defined mainly by an outer surface of the cylinder jacket with the end face, an outer surface of the Luer lock and an outer surface of the closure device. By providing a centrifugation support specially adapted to the container, it is possible for the container to undergo centrifugation in a manner that is particularly gentle on the material involved.

In an alternative development, a complete system with at least one first system for producing at least one biologically active substance, and with at least one second system for producing at least one biologically active substance, is proposed, the first system differing from the second system in terms of at least one means. Here, “first system” defines, for example, a system for treatment of a human, and “second system” defines, for example, a system for treatment of a large mammal, for example a horse, cow and/or camel. The system for humans has, for example, a 50-ml syringe and a cannula and/or a butterfly for blood sampling, a disposable sterilization swab for disinfecting the puncture site and the pierceable membrane, a connection tube for delivering the blood into the container, the container with a Luer lock, a closure cap for closing the Luer lock, a centrifugation support for the centrifugation of the container, and several, preferably five, 10-ml syringes and at least one cannula for aliquoting the collected blood serum. By contrast, in the system for a large mammal, such as a horse, the sterilization swab can be omitted, for example, since the immune system of a horse is less susceptible to infections of the puncture site, or the system could include two 50-ml syringes, two containers and/or a suitable number of aliquot pipettes (for example ten pipettes holding 10 ml, five holding 20 ml or two holding 50 ml), since the blood volume of a horse is about five to ten times greater that that of a human, and a greater volume of blood can accordingly be withdrawn. Thus, for each order and/or genus of mammal to be treated, for example human, monkey, dog, cat, perissodactyl, artiodactyl, proboscoid, a specially adapted system can be provided which is adapted specifically to the treatment needs of the human and/or animal.

A complete system could also be provided that comprises various systems which differ in terms of at least one means and which are designed for different countries in which the device is marketed and are adapted to the conditions existing in these countries, for example the language that is spoken there and/or the temperatures that prevail there. Such a means could, for example, be the operating instructions and/or a cooling system.

The invention also relates to a method for using a device, in which method blood constituents are firstly withdrawn from an animal organism using a blood-sampling device, after which, secondly, at least some of these blood constituents are delivered to at least one container, and, thirdly, at least some of the blood constituents can be exposed, in a cavity of the container, to at least one stress situation in order to produce at least one biologically active substance. Here, a “stress situation” is to be understood in particular as an event that departs from the normal in vivo conditions of the blood constituents or blood cells. This event can be a temperature change, a pressure change, a change in a chemical potential, a change in an electrical potential, a pH change, a change in an electrolyte concentration, a mechanical stress, such as shearing, and/or another event that appears useful to a person skilled in the art.

It is also conceivable for the method to include other steps, for example incubation and/or movement and/or agitation of the container with the blood present therein, centrifugation of the container for separation of the blood constituents and at least one shearing body, collecting of the biologically active substance, for example enriched in one blood constituent, in the form of a removal of aliquots from the cavity of the container, preferably via the pierceable membrane, storage of the collected aliquots and/or other steps that appear expedient to a person skilled in the art. Additional intermediate steps, for example pre-treatment of the blood before delivery to the container, are possible between the method steps listed above. With the method described, a biologically active substance can be produced by simple structural means, safely and efficiently.

The device is intended for approval and use according to the guidelines of the German Medicinal Products Act.

### BRIEF DESCRIPTION OF THE DRAWINGS

The accompanying figures, in which like reference numerals refer to identical or functionally similar elements throughout the separate views and which, together with the detailed description below, are incorporated in and form part of the specification, serve to further illustrate various embodiments and to explain various principles and advantages all in accordance with the present invention. The drawings, the description and the claims contain numerous features in combination. A person skilled in the art will advantageously consider the features singly and will bring them together to give further useful combinations.

FIG. 1 is a sectional view of a container;

FIG. 2 is a plan view of the container of FIG. 1;

FIG. 3 is a diagrammatic view of a system, with the container of FIG. 1, for a treatment of a human;

FIG. 4 is a diagrammatic view of a system, with the container of FIG. 1, for a treatment of a horse;

FIG. 5 is a diagrammatic view showing a complete system with a first system and second system according to FIGS. 3 and 4;

FIG. 6 is a sectional view of a centrifugation support for a container according to FIG. 1;

FIG. 7 is a sectional view showing the centrifugation support of FIG. 6 in which the container of FIG. 1 is received;

FIG. 8 is a flow chart of a method sequence;

FIG. 9 is a diagrammatic view, partially in section, showing a process of blood delivery using the container of FIG. 1; and

FIG. 10 is a diagrammatic view, partially in section, showing a process of blood serum removal using the container according to FIG. 1.

### DETAILED DESCRIPTION OF THE PREFERRED EMBODIMENTS

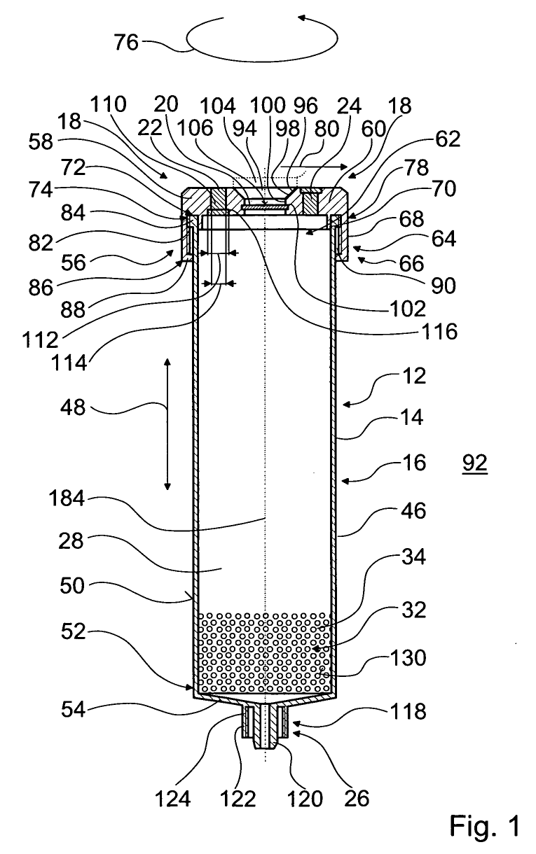

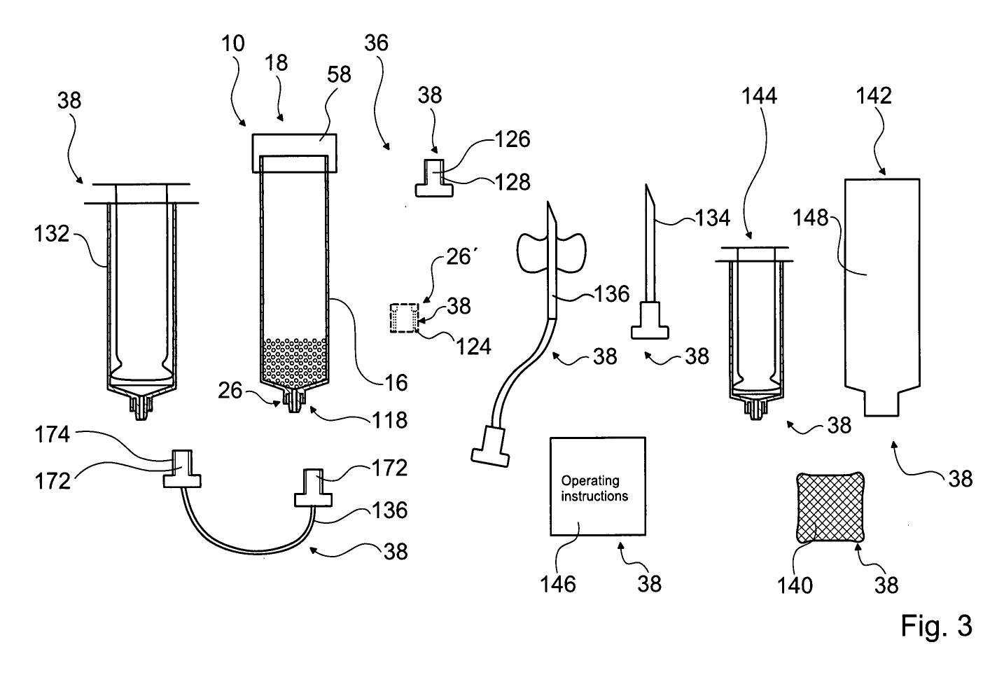

FIG. 1 shows a device 10 for producing at least one biologically active substance. The device 10 has a unit 12, which has a main body 14 made of plastic. The unit 12 is a container 16, which has a wing-free and pistonless design and is accordingly different than a syringe, such as a blood-sampling syringe 132 (see FIG. 3). The main body 14 has a rigid receptacle wall in the form of a cylinder jacket 46, which extends along an axial main direction of orientation 48 of the container 16, and its cylinder jacket surface 50 encloses a cavity 28, which is provided for the sterile storage of blood constituents 30. Arranged at one end 52 of the cylinder jacket 46 of the main body 14 there is an end face 54, which is designed as a bottom part that narrows conically toward a center axis 184 of the main body 14. This end face 54 is formed in one piece with the cylinder jacket 46.

The device 10 or container 16 also has a closure device 18 which is connected irreversibly to the main body 14 and which is arranged at another end 56 of the cylinder jacket 46 of the main body 14. The closure device 18 forms a cover in the form of a lid 58 made of metal. The closure device 18 or lid 58 has a cover area 60, which extends perpendicular to the main direction of orientation 48 of the container 16 and which completely covers an opening 62 of the cavity 28. The closure device 18 also has a connection area 64 which extends, in an overlap area 66 between the closure device 18 and the main body 14, parallel to the main direction of orientation 48 of the container 16 and to the cylinder jacket 46.

The connection area 64 is formed by two ring segments 68, 70 which extend in the main direction of orientation 48 of the container 16 and which form a receiving area 72 for an end area 74 of the main body 14. The ring segment 68 extends in the circumferential direction 76 about an outer surface of the cylinder jacket 46, and the ring segment 70 extends in the circumferential direction 76 in the inner surface of the cylinder jacket 46. On an end 78 of the ring segment 68 directed toward the cover area 60 or adjoining the cover area 60, there is a contour 82 in the form of a groove that extends out from the cylinder jacket 46 in the radial direction 80 and into which there engages a locking element 84 in the form of a projection, which is formed integrally on the end area 74 of the cylinder jacket 46 of the main body 14 and which extends in the radial direction 80 out to the ring segment 68. The contour 82 in this case is an inner thread of the closure device 18, which inner thread comes into engagement with the locking element 84 in the form of an outer thread of the main body 14 in a procedure in which the lid 58 is screwed onto the main body 14 upon production of the container 16. In this production process, not shown in detail here, the closure device 18 is fixed inseparably on the main body 14 not only by the form-fit match between the contour 82 and the locking element 84, but also by way of a further method step, for example a thermal connection step.

Formed integrally on an end 86 of the ring segment 68 opposite the end 78, or remote from the cover area 60, there is a projection 88 which extends inward in the radial direction 80 to the cylinder jacket 46 and which closes off a gap 90 formed in the overlap area 66 between the main body 14 and the ring segment 68 of the closure device 18. The projection 88 also seals off the gap 90 from an outer environment 92 or from air situated in the outer environment 92. A filler material, for example an adhesive, can be introduced into the gap 90 in order to increase the airtightness and to maintain sterility in the cavity 28. An adhesive additionally serves to permit the irreversible connection of the closure device 18 to the main body 14.

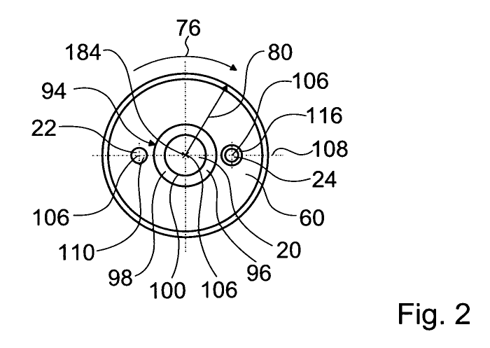

The device 10 or container 16 also has a pierceable membrane 20, which is arranged centrally in the cover area 60 of the closure device 18 (see FIG. 2). The pierceable membrane 20 is formed of a rubber layer and, in the state when not pierced, is impermeable to solids, liquids and/or gases. For this purpose, an opening 94 is formed in the cover area 60 and has a boundary 96 that forms a conically extending portion 98 and a portion 100 extending parallel to the main direction of orientation 48. In the parallel portion 100, there is a cutout 102 which is directed outward in the radial direction 80 and which holds the pierceable membrane 20 in its position perpendicular to the main direction of orientation 48. Moreover, the closure device 18 can have a protection unit 104 (shown here by broken lines) in the form of a pull-off film with pull-off tab made of plastic, which is intended to keep the pierceable membrane 20 sterile until it is used.

The device also has a gas outlet valve 22 and a gas inlet valve 24, which are arranged in the closure device 18 and which are designed as nonreturn valves with diaphragm. The gas outlet and gas inlet valves 22, 24 each have a center point 106 which, with a center point 106 of the opening 94 lying on the center axis 184 of the container 16, lie on an axis 108, the pierceable membrane 20 being arranged along the axis 108 centrally between the gas outlet valve 22 and the gas inlet valve 24 (see FIG. 2). The gas outlet and gas inlet valves 22, 24 have a cylinder-shaped portion 110 extending along the main direction of orientation 48 of the container 16, and a disk-shaped portion 116 whose diameter 112 is wider than a diameter 114 of the cylinder-shaped portion 110. The membrane (not shown in detail here) for controlling the movement of air into and out of the cavity 28 is arranged in the cylinder-shaped portion 110. Along the main direction of orientation 48, the gas outlet and inlet valves 22, 24 span the cover area 60 of the closure device 18 along its full axial length. The gas outlet and inlet valves 22, 24 are of identical construction and are introduced in reverse orientations into the closure device 18. They are in contact with the cavity 28, and an exchange of gas or air can take place, through the gas outlet and inlet valves 22, 24, between the cavity 28 and an outer environment 92 of the container 16.

A connection unit 26 is also arranged on the main body 14. The connection unit 26 is designed in one piece with the end face 54, which is arranged at the end 52 of the cylinder jacket 46 directed away from the closure device 18, and it is arranged centrally in the radial direction 80 in said end face 54. Moreover, the connection unit 26 is formed by a Luer lock 118, which is composed of a Luer cone 120 and a wall 122 or a sleeve nut with an integrally formed thread in the form of an inner thread 124. A closure cap 126 with an outer thread 128 can, for example, be screwed onto this inner thread 124 in order to close the container 16 (see FIG. 3).

The cavity 28 has a capacity of greater than 50 ml and, in this illustrative embodiment, a capacity of 60 ml. In order to monitor an operation of filling the device 10 or container 16, a scale (not shown here), for example in 10 ml increments, can be provided on the cylinder jacket surface 50.

The device 10 also has a large number of shearing bodies 32, in particular in this illustrative embodiment 240 such shearing bodies 32, which are arranged in the cavity 28 of the container 16. To make matters clearer, only some of the shearing bodies 32 are shown in FIG. 1. The shearing bodies 32 are formed by beads 34, which have a diameter of 4 mm, are made of glass and have a smooth surface 130. The shearing bodies 32 take up a volume of about 10 ml of the 60 ml volume of the container 16.

FIG. 3 shows a system 36 for producing a biologically active substance using a device 10 in the form of a container 16; the component parts of the system 36 are not depicted true to scale. In addition to the container 16, further component parts of the system 36 are additional means 38, such as a blood-sampling syringe 132, a cannula 134, a butterfly 136, a connection tube 138, two disposable sterilization swabs 140, a closure cap 126, a centrifugation support 142, five aliquot syringes 144, and operating instructions 146, for sampling of blood and/or preparation of blood. Each means 38 is shown only once. As an alternative to the one-piece design of the Luer lock 118 with the container 16, a separate connection unit 26′, shown here by broken lines, could also be provided, designed as an attachable sleeve nut with inner thread 124. The system 36 shown is designed for a treatment of a human.

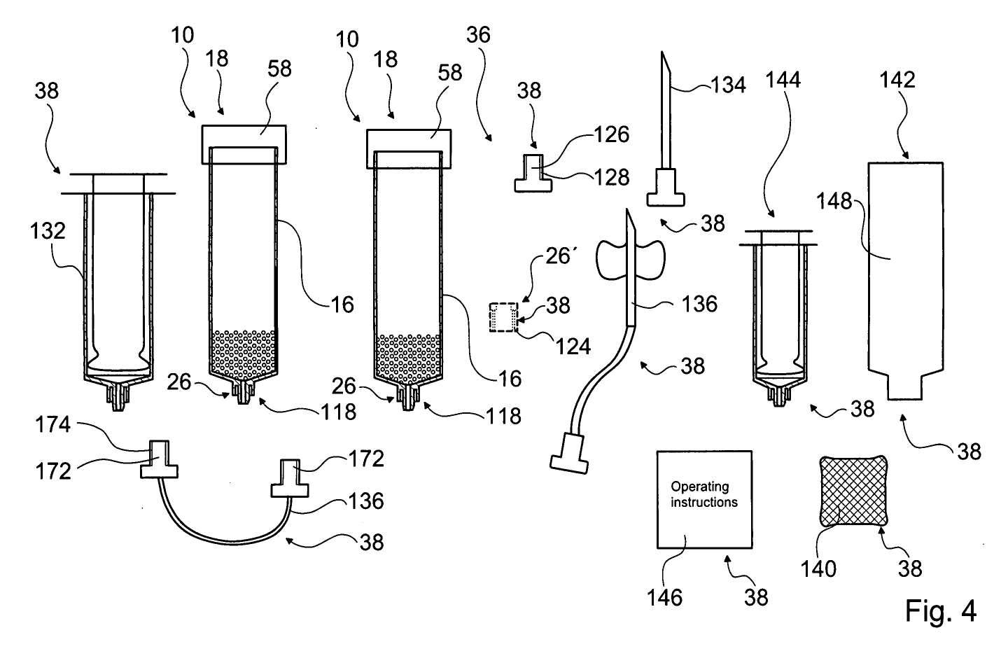

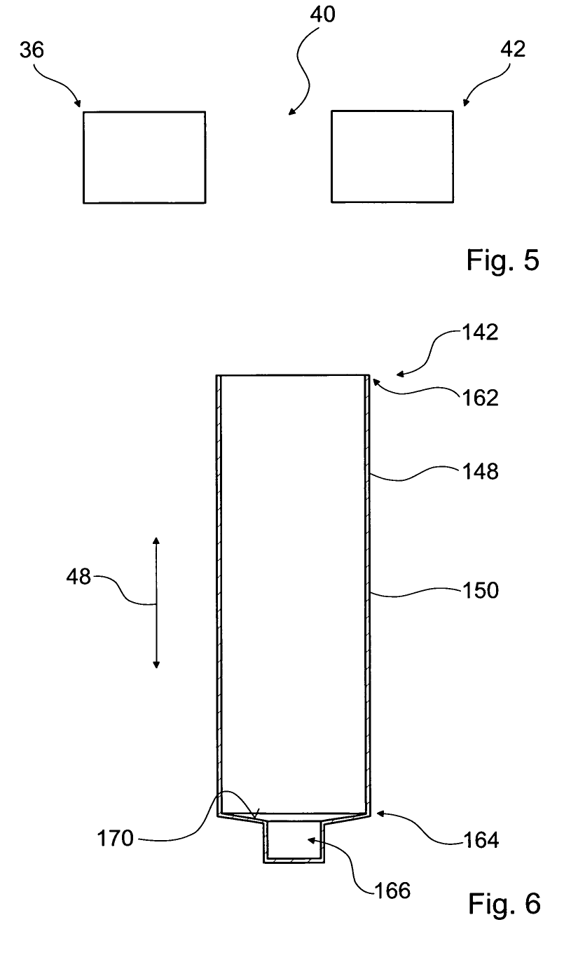

By contrast, FIG. 4 shows a second system 42 for the treatment of a horse. This second system 42 differs from the first system 36 in terms of several means 38. The two systems 36, 42 thus form a complete system 40 (see FIG. 5). The second system 42 has two containers 16, two blood-sampling syringes 132 (of which only one is shown) and ten aliquot syringes 144 (of which only one is shown), and it is therefore designed to prepare twice the amount of blood compared to the system 36 for humans.

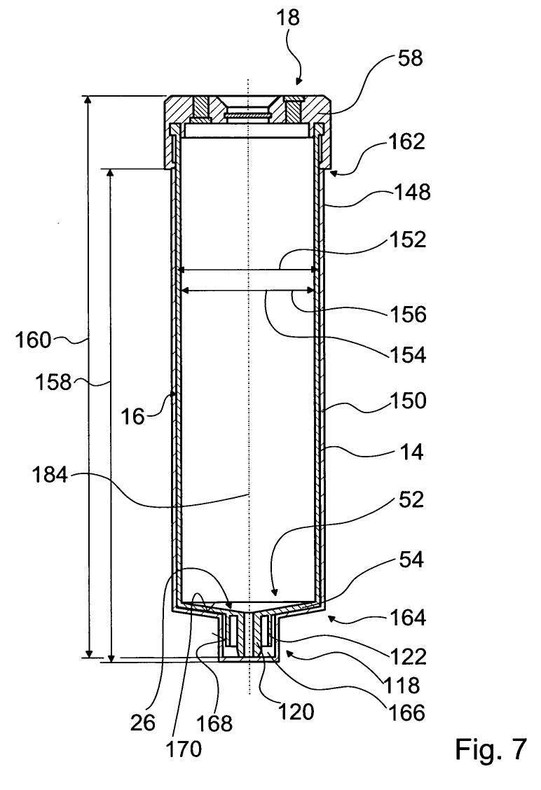

A centrifugation support 142 specially designed for the container 16, and in the form of a centrifugation tube 148, is shown in FIG. 6. The shape of the centrifugation support 142 is adapted to an outer shape of the container 16. The centrifugation tube 148 here has a main body 150 which, like the main body 14 of the container 16, extends in the main direction of orientation 48 of the container 16. A diameter 152 of the main body 150 is wider than a diameter 154 of the main body 14, such that the container 16 in terms of its width 156 is fully received by the centrifugation support 142. An axial length 158 of the main body 150 is shorter than an axial length 160 of the main body 14, such that, when the container 16 is inserted into the centrifugation support 142, the closure device 18 or lid 58 protrudes beyond an end 162 of the centrifugation support 142 (see FIG. 7). At an end 164 of the centrifugation tube 148 remote from the end 162, a receiving seat 166 is integrally formed for the end 52 or end face 54 or connection unit 26 or Luer lock 118 and closure cap 126. This receiving seat 166 is of such a configuration that an outer contour 168, formed by the elements 26, 54, 118, 126 at the end 52 of the container 16, bears stably on an inner contour 170 of the receiving seat 166.

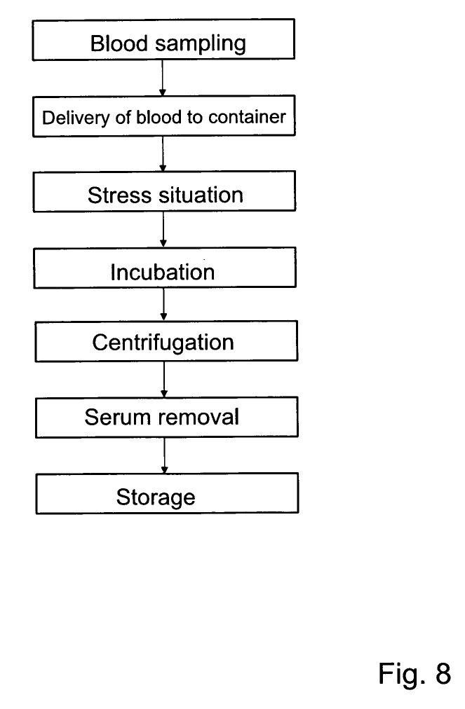

A method for use of a device 10 and of a system 42 is described below, and, although the system 42 provides materials for several procedures, only one blood-sampling and preparation procedure is described here. The method is shown as a flow chart in FIG. 8.

Before the device 10 or container 16 is used, a blood-sampling device 44 is used to withdraw blood or blood constituents 30 from a patient or animal organism, for example a horse. In a procedure known to persons skilled in the art, a volume of 50 ml of blood (not shown) is withdrawn from the patient using a blood-sampling syringe 132 and an attached cannula 134 or butterfly 136, at a puncture site that has first been disinfected using a disposable sterilization swab 140.

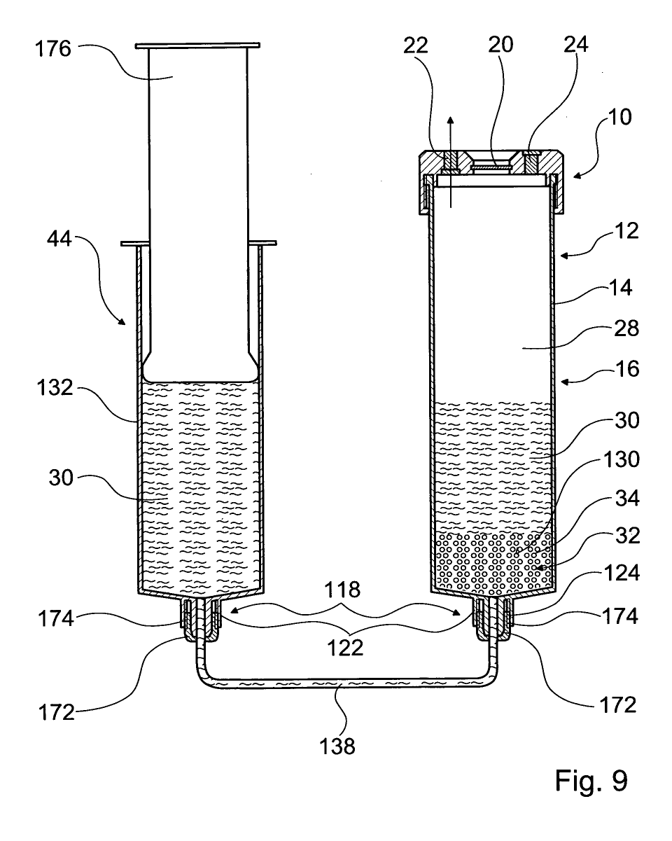

These blood constituents 30 are then delivered to the container 16, for which purpose a connection tube 138 with two coupling positions 172 is used. Prior to the delivery, the cavity 28 of the container 16 holds only the shearing bodies 32 and air (not shown). The connection tube 138 is screwed, with the aid of an outer thread 174 at each of the coupling positions 172, onto the inner thread 124 of the Luer lock 118 of the blood-sampling syringe 132 and, respectively, of the Luer lock 118 of the container 16. The blood constituents 30 are then forced through the connection tube 138 into the container 16 by application of a pressure to a piston 176 of the blood-sampling syringe 132, in which process the air present in the cavity 28 is able to escape via the gas outlet valve 22 (see FIG. 9). During this delivery, some of the blood constituents 30 in the cavity 28 of the container 16 are exposed to a stress situation in order to produce a biologically active substance, by means of the blood cells in the blood being forced past the shearing bodies 32 at high pressure by the filling pressure during delivery of the blood. This interaction of the blood cells with the beads 34 and with their smooth surface 130 signifies, for the blood cells, a stress situation that simulates an inflammatory process. As a reaction to this stimulation, the blood cells build products of metabolism (interleukin-1, and interleukin-1 receptor antagonist) which occur in inflammatory reactions.

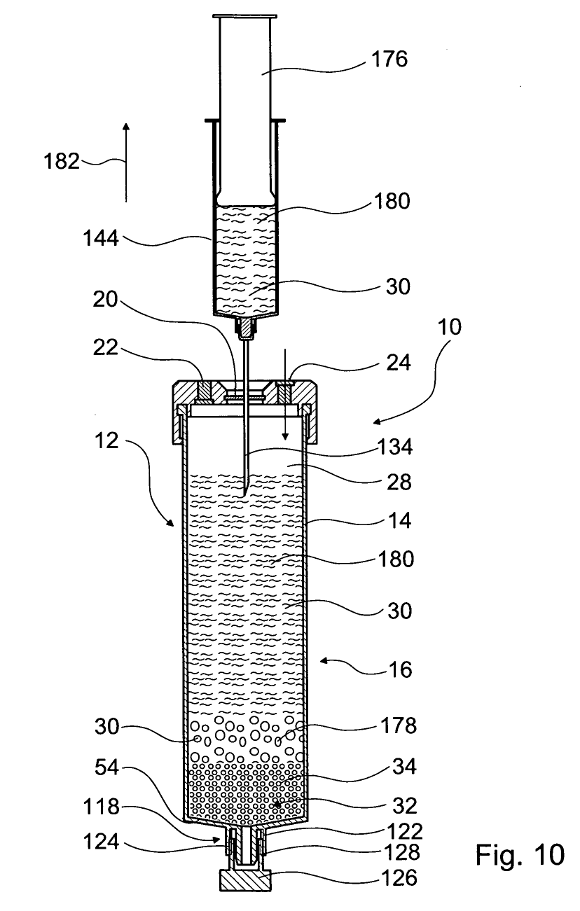

After removal of the connection tube 138, a closure cap 126 is screwed with its outer thread 128 onto the inner thread 124 of the Luer lock 118 in order to close the container 16 (see FIG. 10).

To achieve a sufficient enrichment of the blood with these products of metabolism, preferably enrichment with the anti-inflammatory interleukin-1 receptor antagonist, the withdrawn blood located in the device 10 is incubated for 2 to 8 hours in an incubator at 37° C. In principle, however, it would also be possible to dispense with this incubation and replace it, if appropriate, by an equivalent technique, for example agitation of the container 16.

After the incubation time, the device 10 is removed from the incubator and is centrifuged at room temperature in the centrifugation support 142 by a centrifuge, for a length of time that is sufficient for sedimentation of the shearing bodies 32 and solid blood constituents 30 and coagulum 178 (for example 10 minutes at 5000 rpm). After the centrifugation time, three layers have formed. The heavy shearing bodies 32 have settled first on the end face 54 of the container 16, followed by the cellular blood constituents 30 and coagulum 178, and, finally, the liquid blood constituents 30 or blood serum 180 (see FIG. 10).

Thereafter, as is likewise shown in FIG. 10, the blood serum 180 is removed via a pierceable membrane 20. For this purpose, the pierceable membrane 20 is disinfected using a disposable sterilization swab 140 (not shown), and a cannula 134 of an aliquot syringe 144 is guided through the pierceable membrane 20 and into the cavity 28 of the container 16 filled with blood serum 180, until the cannula 134 is immersed in the blood serum 180. By pulling a piston 176 of the aliquot syringe 144 back in the axial direction 182, the liquid blood constituents 30 and blood serum 180 are now removed from the cavity 28 of the container 16. To make the removal of serum easier, the cavity 28 is filled with air via the gas inlet valve 24 for pressure equalization. The removal step is repeated until all of the blood serum 180 has been removed from the cavity 28. The aliquot syringes 144 thus obtained, with the patient's blood serum 180 enriched with interleukin-1 receptor antagonist, can now be used to return the blood serum 180 to the patient. For this purpose, they can be stored for a period of up to 7 months at a temperature of from −18 to −20° C.

This disclosure is intended to explain how to fashion and use various embodiments in accordance with the invention rather than to limit the true, intended, and fair scope and spirit thereof. The foregoing description is not intended to be exhaustive or to limit the invention to the precise form disclosed. Modifications or variations are possible in light of the above teachings. The embodiments were chosen and described to provide the best illustration of the principles of the invention and its practical application, and to enable one of ordinary skill in the art to utilize the invention in various embodiments and with various modifications as are suited to the particular use contemplated. All such modifications and variations are within the scope of the invention as determined by the appended claims, as may be amended during the pendency of this application for patent, and all equivalents thereof, when interpreted in accordance with the breadth to which they are fairly, legally, and equitably entitled.

1. A device which is used for producing at least one biologically active substance and which has at least one unit with a main body, wherein the unit is a container.

2. The device according to claim 1, wherein the container has a wing-free design.

3. The device according to claim 1, wherein the container has a pistonless design.

4. The device according to claim 1, wherein at least one closure device is connected irreversibly to the main body.

5. The device according to claim 1, including at least one pierceable membrane.

6. The device according to claim 1, including at least one gas outlet valve or one gas inlet valve.

7. The device according to claim 1, wherein at least one connection unit is arranged on the main body.

8. The device according to claim 1, including at least one cavity, which is provided for sterile storage of blood constituents.

9. The device according to claim 1, including at least one shearing body.

10. The device according to claim 1, including
at least one cavity, which is provided for sterile storage of blood constituents; and
at least one shearing body

11. The device according to claim 10, wherein the at least one shearing body is arranged in the at least one cavity.

12. The device at least according to claim 9, wherein the at least one shearing body is formed by a bead.

13. The device at least according to claim 8, wherein the at least one cavity has a capacity of greater than 50 ml.

14. A device according to claim 1, wherein the device forms part of a system for producing at least one biologically active substance, wherein the system includes at least one further means for blood sampling and/or blood preparation, wherein said means is provided, in addition to the container, as a component of the system.

15. A device according to claim 14, including at least one first system for producing at least one biologically active substance and at least one second system for producing at least one biologically active substance, wherein the first system differs from the second system in terms of at least one means.

16. A method for using a device, wherein the device is for producing at least one biologically active substance and has at least one container with a main body, wherein the method comprises:
withdrawing blood constituents from an animal organism using a blood-sampling device;
delivering at least some of said blood constituents to at least the container; and
exposing at least some of said blood constituents in a cavity of said container to at least one stress situation to produce at least one biologically active substance.
